(19) United States
(12) Patent Application Publication
(10) Pub. No.: US 2024/0050000 A1
(43) Pub. Date: Feb. 15, 2024

(54) SAMPLE-RECEIVING DEVICE

(71) Applicant: Anvajo GmbH, Dresden (DE)

(72) Inventors: Aitor EZKERRA FERNANDEZ, Dresden (DE); Daniel HAMMER, Dresden (DE)

(21) Appl. No.: 18/260,945

(22) PCT Filed: Jan. 11, 2022
(86) PCT No.: PCT/EP2022/050447

§ 371 Date: Jul. 11, 2023

(30) Foreign Application Priority Data
Jan. 12, 2021 (DE) 10 2021 200 214.5
Dec. 20, 2021 (DE) 10 2021 214 685.6

(51) Int. Cl. A61B 5/15; B08B 1/00

(52) U.S. Cl. CPC A61B 5/150343; B08B 1/006

(57) ABSTRACT

A sample receiving device may have a first part and a second part, the first part may have at least two rod-shaped sample receiving units which are connected to one another via a bendable connection. The second part may have a locking device such that the bendable connection is bent by the shape of the locking device and the ends of the at least two sample receiving units can be guided toward each other so that the sides of the at least two sample receiving devices facing in the direction of a sample are arranged directly next to one another, are locked in this position by the locking device and a single sample can be received in the at least two sample receiving units or with the at least two sample receiving units.

The present application relates to a sample receiving device and a sample receiving method.

In the field of sample processing, it is a challenge, especially with liquid samples that are to be taken up and analyzed in small quantities, to quickly take up and further process a suitable number of samples. A gain in efficiency is possible here by using so-called “aliquots”, i.e. by using partial samples without negatively affecting the quality of the analysis. For example, U.S. Pat. No. 5,741,412 A discloses an arrangement with multiple capillaries for sample collection and analysis. A disadvantage of such systems is the rigid arrangement, which allows continuous flow but limits the flexibility of the analysis.

The present invention is therefore based on the object of proposing a sample receiving device which avoids the disadvantages mentioned, i.e. with which a sample collection can be carried out quickly and, in addition, the further analysis can be handled flexibly.

This object is solved according to the invention by a sample receiving device according to the main claim and a method according to the independent claim. Advantageous embodiments and further developments are described in the dependent claims.

In one embodiment, the sample receiving device has a first part and a second part. The first part has at least two rod-shaped sample receiving units connected to one another via a bendable connection, the ends of which facing away from the bendable connection are spatially spaced apart from one another in a basic state. The second part has a locking device which is designed in such a way that, when the first part is placed with its side facing away from the sample receiving units onto the second part, the bendable connection is bent by the shape of the locking device and the ends of the at least two sample receiving units can be guided towards each other, so that the sides of the at least two sample receiving units facing in the direction of a sample are arranged directly next to each other, are locked in this position by the locking device and a single sample can be received in the at least two sample receiving units or with the at least two sample receiving units.

The fact that the sample receiving units are at a defined distance from each other in the basic state and are not guided towards each other at their tips or ends, i.e. at the sides facing the sample to be received, until the appropriate translational movement, i.e. the joining of the first part and the second part or the placing of the first part on the second part, provides a clear spatial separation, which nevertheless enables rapid sample reception after the movement has been carried out. The bendable or articulated connection of the sample receiving units, which can also be of flexible design, i.e. bendable and extensible, allows the sample receiving units to be moved in a reproducible manner. Guiding the ends or tips of the sample receiving units towards each other is typically achieved by angling the sample receiving units.

The first part may also be referred to as the upper part and the second part as the lower part, but it is also possible to refer to the first part as the lower part and the second part as the upper part. The ends of the sample receiving units pointing away from the bendable connection are usually oriented downward, i.e. toward the center of the earth. The individual sample picked up by the sample receiving units (i.e. the sample receiving units are designed to pick up a sample) may be picked up entirely by the sample receiving units, but it may also be provided to pick up only partial volumes of the sample. The second part may comprise only the locking device, but it may also be provided that, in addition to the locking device, other elements are part of the second part of the sample receiving device, for example a holding unit for gripping and holding the second part.

The tips of the sample receiving units or the ends of the sample receiving units are preferably in direct contact with each other after the first part has been placed on the second part and the bendable connection has been bent or angled. In this context, the term “direct contact” should be understood to mean not only a distance of 0 mm but also a distance of up to 2 mm, typically a small distance of 0.1 mm to 1 mm, between the tips of the sample receiving units.

It may be provided that the bendable connection, which may also be referred to as an articulated connection, is a hinge-shaped connection. Alternatively, this connection can also be designed as an articulated connection, preferably as a solid-state articulated connection or swivel joint connection. Typically, the sample receiving units and the connection are designed as a material bond, i.e. as a single component or one-piece. Alternatively, however, the sample receiving units can each exist as individual parts and be brought into contact with and connected to one another by a form-fit connection. Furthermore, it can also be provided that there is an adhesive connection between the sample receiving units designed as individual units, so that they are connected to each other by the adhesive connection. This creates a connection that is easy to make and easy to modify.

The bendable connection may be located centrally between the two sample receiving units or, if more than two sample receiving units are provided, centrally between two adjacent sample receiving units. This results in a uniform bending. However, provision may also be made to allow asymmetrical bending, for example by placing the bendable connection off-center between two of the sample receiving units.

In one embodiment, the sample receiving device has an upper part and a lower part that can be connected to each other in a form-fitting and/or force-fitting manner. The upper part comprises a holding housing, wherein at least two rod-shaped sample receiving units or at least two bendable sample receiving units are arranged on the holding housing and connected thereto in each case via a bendable connection, which sample receiving units are arranged spatially spaced apart from one another in a basic state. The lower part has a locking device which is designed in such a way that the at least two sample receiving units can be guided towards each other either by an external force or by the locking device of the lower part, so that the sides of the at least two sample receiving units which are arranged facing in the direction of a sample are arranged directly next to each other, are locked in this position by the locking device and a single sample can be received in the at least two sample receiving units or with the at least two sample receiving units. The first part thus corresponds to the upper part, the second part corresponds to the lower part, but in further embodiment examples the first part can also correspond to the lower part and the second part to the upper part.

The fact that the sample receiving units are at a defined distance from each other in the basic state and are not guided towards each other at their tips, i.e. on the sides facing away from the holding housing, until the appropriate translational movement of the lower part in the direction of the upper part or of the upper part in the direction of the lower part is performed, provides a clear spatial separation which nevertheless permits rapid sample receiving units after the movement has been performed. The bendable connection of the sample receiving units to the holding housing, which can also be of flexible design, i.e. bendable and expandable, allows the sample receiving units to be moved in a reproducible manner. In that the upper part and the lower part are connected or connectable to one another in a form-fitting and/or force-fitting manner, a compact and mechanically stable arrangement is also provided which can execute the described movement as desired.

In this context, the term “bendable” is intended to mean in particular an embodiment of the invention in which a bending radius before breaking is 0.25° to 180°, preferably 0.25° to 90°, and elastic, plastic, largely elastic or largely plastic deformation of the sample receiving units takes place with a small irreversible plastic or elastic deformation component. It may also be provided that the sample receiving units are separable from the holding housing by bending at the junction, which is typically web-shaped, beyond the respective bending radius so that the junction breaks. Typically, the connection point is designed in such a way that when the lower part is pulled off the upper part, the sample receiving units are moved back to their original position, i.e. the position before the lower part was put on, or a position in which the sample receiving units are further apart than in the original position or the position in which the sample receiving units were brought together, so that they can be separated more easily.

The term “rod-shaped” is to be understood in the context of this writing in particular as meaning that a length of the element designated thereby is at least 10 percent greater than its width and/or depth. Especially cylindrical elements or hollow cylindrical elements shall be described by the term “rod-shaped”.

It may be provided that the lower part has a housing provided with an opening serving as a locking device. The sample receiving units can thus be spatially fixed in a defined manner through the opening. The lower part is typically disc-shaped or ring-shaped.

The sample receiving units can be inserted into the opening of the housing and moved relative to each other by the external force in such a way that the sample receiving units are locked in the opening serving as a locking device after insertion to allow the sample receiving devices to be held securely in a defined position.

The lower part can have a housing provided with a through opening serving as a locking device, which housing has at least one tapered part, wherein the lower part can be placed on the at least two sample receiving units in a translational movement in such a way that the at least two sample receiving units, when inserted into the tapered part of the lower part, are guided towards each other by the tapered shape on their sides arranged facing in the direction of a sample. It may also be provided that the lower part has a rectilinearly extending part. Due to the tapered part of the lower part, which, if provided and if the lower part is not only formed of a part tapering in the direction of the opening, has a greater spatial distance from the holding housing than the rectilinearly extending part of the lower part, guidance of the sample receiving units is also ensured. The fact that the tips of the sample receiving units are usually in direct contact, i.e. in direct contact, with each other after the translational movement has been performed, typically at least in the area of the tips, means that they can be guided through the opening of the lower part and also take up comparatively small sample volumes. In this context, the term “direct contact” should be understood to mean not only a distance of 0 mm but also a distance of up to 2 mm, typically a small distance of 0.1 mm to 1 mm, between the tips of the sample receiving units.

The lower part may have a conical tapered portion, typically a conical tapered bottom portion, to provide uniform guidance of the tips of the sample receiving units.

It may be provided that the rectilinearly extending part of the lower part is insertable into a corresponding recess of the upper part for establishing operational readiness of the at least two sample receiving units during the translational movement. This realizes a compact design of the sample receiving device, in which the translational movement can also be guided specifically by holding the lower part. The rectilinearly extending part of the lower part may also be configured to also taper in the direction of the tapered portion, although the taper is less than that of the tapered portion of the lower part. A taper of up to 5° is to be considered “straight” in this context.

A length of the rectilinearly extending part of the lower part can correspond to a length of the sample receiving units, so that in the basic state the sample receiving units in the lower part or lower part are arranged at a distance from each other and, at the start of the translational movement, are immediately brought together with their tips by contact with the tapered part. Alternatively or additionally, the length of the sample receiving units can be selected so that the side of the sample receiving units facing in the direction of the sample is located in the region of the opening provided on the lower part. In this context, the area of the opening is to be understood in particular as the area that is at a distance from the opening that is less than the length of the opening.

The lower part is typically movably guided in a guide arranged in the holding housing of the upper part and is preferably limited in its ability to move by at least one stop. This results in a clearly defined movement to transfer the sample receiving device from the basic state, in which the upper part and lower part are not pushed together, to the pushed-together state. It can also be provided that the lower part and the upper part are connected to each other by a plug-in connection. Typically, the lower part and the upper part are made of an identical material, but it is also possible to use different materials for the lower part and the upper part. In addition, the first part and the second part of the lower part can be the same length.

It can also be provided to use a holding device, typically designed in two parts, as a locking device by which the at least two sample receiving units moved by the external force are clamped in their position. For this purpose, the holding device is preferably attached to or in the connections.

It may be provided that at least one of the sample receiving units is in the form of a capillary, a tube, a fiber, preferably a hollow fiber, a swab, a brush, a spatula, a spatula knife, a needle, a membrane, a scoop, a spoon, a sponge, preferably a solid sponge, or a rod, preferably a helical rod, or with a capillary, a tube, a fiber, preferably a hollow fiber, a swab, a brush, a spatula, a spatula knife, a needle, a membrane, a scoop, a spoon, a sponge, preferably a solid sponge, or a rod, preferably a helical rod. Thus, different types of sample reception are possible, which can be adapted to the respective application. The sample itself can be in liquid form, as a solid, but also be a gel. The rod-shaped or elongated sample receiving units allow the sample to be moved in the direction of the opening, while at the same time leaving sufficient volume to accommodate the sample.

The upper part or the first part can have exactly three sample receiving units arranged in a row arrangement, of which the sides facing away from the holding housing of the two sample receiving units arranged at the edges are arranged in a first plane in the basic state and the side facing away from the holding housing of the centrally arranged sample receiving unit is arranged in a second plane, different from the first plane and closer to the holding housing, in the basic state with respect to the two sample receiving units arranged at the sides. In particular, if the length of the sample receiving units is adapted accordingly, a compact, space-saving arrangement can be achieved in which all the sample receiving units end in one plane, i.e. at the same height, after the translational movement has been performed, i.e. in the pushed-together state. In particular, any arrangement in which a triangle is formed in plan view of the tips, i.e. on the sides facing the sample or away from the holding housing, in which the largest internal angle is at least 120°, is to be regarded as a row arrangement in this context. Thus, it can be provided both that all three sample receiving units are arranged in one plane in lateral view and that at least one of the sample receiving units is positioned in a plane offset from the plane of the other two sample receiving units.

The lower part can have a volume-variable cleaning element covering the opening, preferably a sponge, into which the at least two sample receiving units can be inserted. Alternatively, a volume-variable cleaning element covering the opening can be placed on the at least two sample receiving units. Thus, cleaning of at least the outer sides of the sample receiving units can be performed after each sample collection. The cleaning element is typically displaced with the lower part and thus slides along the outer sides of the sample receiving units. Ideally, these outer sides are run down once lengthwise. The cleaning element can be made of polyvinyl chloride (PVC), typically PVC foam, polyurethane (PU), typically PU foam, acrylonitrile butadiene rubber foam, ethylene vinyl acetate (EVA) foam, soft polyethylene (low-density polyethylene, LDPE), typically LDPE foam, neoprene, expanded polystyrene, silicone rubber foam, polypropylene foam, polyterephthalate foam, cellulose, nitrocellulose or cotton may be formed or at least comprise said materials.

The opening itself is preferably elongated, i.e. its length is greater than its width. This makes it easier to insert the sample receiving units into the opening. Alternatively, the opening can be round or have rounded corners.

The holding housing or the first part can be formed closed on one of the sides facing away from the at least two sample receiving units in order to increase the mechanical stability and to prevent an unwanted influence on the sample contained in the sample receiving units or to form a barrier for the received sample.

The locking device of the second part may be funnel-shaped for receiving the first part. Alternatively or additionally, it can be provided that the first part and the second part are or can be connected to each other in a form-fitting or force-fitting manner. The two parts being connected or being able to be connected to each other in a form-fitting or force-fitting manner, also allows for a compact and mechanically stable arrangement that can perform the described movement as desired.

At least the first part together with the at least two sample receiving units can be made of or consist of a plastic that is transparent to electromagnetic radiation in the optically visible wavelength range, i.e. in the wavelength range between 400 nm and 700 nm. “Transmissive” in this case should be understood to mean that at least 90 percent of the incident electromagnetic radiation is transmitted through the component so that a user can visually check a fill level of the sample receiving units. This is particularly advantageous in situations where correct sample collection may be difficult (for example, when taking blood samples from the fingertip). Typically, the first part and the second part are formed from an identical material, but it is also possible to provide that different materials are used for the first part and the second part.

The sample receiving unit may or the upper part or the first part and the lower part or the second part may be formed of or at least comprise a polymer, a metal, rubber, glass or an elastomer. The upper part and the lower part are typically made of the same material, but different materials can also be used for different parts or units. Preferably, the sample receiving unit, i.e. the upper part and the lower part, is made of polypropylene (PP), acrylonitrile-butadiene-styrene copolymer (ABS), polystyrene (PS), cycloolefin copolymer (COC), cycloolefin polymer (COP), polyethylene terephthalate (PET), Polyvinyl chloride (PVC), rigid polyethylene (high-density polyethylene, HDPE) or soft polyethylene (low-density polyethylene, LDPE), polyvinyl chloride (PVC), typically PVC foam, Polyurethane (PU), typically PU foam, acrylonitrile butadiene rubber foam, ethylene vinyl acetate (EVA) foam, low-density polyethylene (LDPE), typically LDPE foam, neoprene, expanded polystyrene, silicone rubber foam, polypropylene foam, or polyterephthalate foam, or at least comprise any of said materials. Typically, however, only the lower part is formed from a foam, usually one of the foams mentioned above, or a foam and one of the polymers mentioned, or consists of foam or foam and one of the polymers mentioned, i.e. the upper part can preferably be produced or is produced from the polymer materials mentioned with the exception of the foams.

The invention also relates to an embodiment example of a method for sample receiving by means of the described sample receiving device, in which the at least two sample receiving units are guided towards each other either by an external force or by the locking device of the lower part, so that the sides of the at least two sample receiving units facing in the direction of a sample are arranged directly next to each other, are locked in this position by the locking device, and a single sample is picked up in the at least two sample receiving units or with the at least two sample receiving units.

In a sample receiving method, in particular the lower part of the described sample receiving device can be moved with respect to the upper part in such a way that the at least two sample receiving units are inserted into the tapered part of the lower part and sides of the at least two sample receiving units arranged facing a sample are guided towards each other, so that the sides of the at least two sample receiving units arranged facing a sample are arranged directly next to each other and a single sample is picked up in the at least two sample receiving units.

The invention also relates to an embodiment of a method for sample collection by means of the described sample receiving device, in which the bendable connection is bent by the shape of the locking device when the first part is placed with its side facing away from the ends of the sample receiving devices onto the second part, and the ends of the at least two sample receiving units are guided towards each other, so that the ends of the at least two sample receiving units arranged pointing in the direction of a sample are arranged directly next to each other, are locked in this position by the locking device and a single sample can be received in the at least two sample receiving units or with the at least two sample receiving units.

Examples of embodiments of the invention are shown in the drawings and are described below with reference to FIGS. 1 to 23.

There are shown:

FIG. 1 a sectional view of an upper part of a sample receiving device;

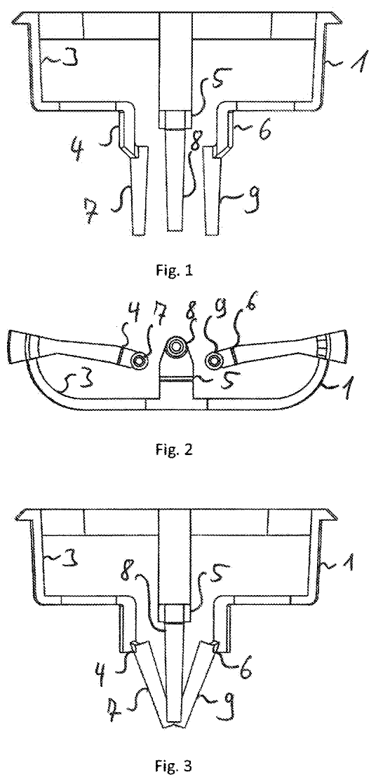

FIG. 2 a top view of the upper part shown in FIG. 1;

FIG. 3 a diagram of the upper part corresponding to FIG. 1 with the sample receiving units brought together;

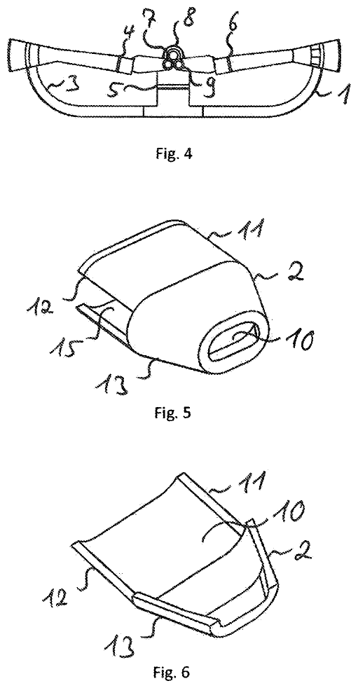

FIG. 4 a view corresponding to FIG. 2 of the condition shown in FIG. 3;

FIG. 5 a perspective view of a lower part;

FIG. 6 a sectional view of the lower part;

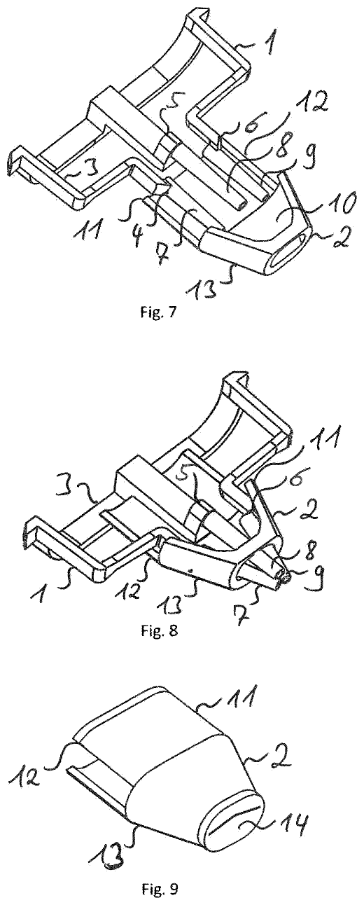

FIG. 7 a sectional view of the upper part combined with the lower part;

FIG. 8 a view corresponding to FIG. 7, in which the upper part and the lower part are pushed into each other;

FIG. 9 a view corresponding to FIG. 5 with a cleaning element;

FIG. 10 a view corresponding to FIG. 8 with the cleaning element

FIG. 11 a view corresponding to FIG. 7 with a disc-shaped or ring-shaped lower part;

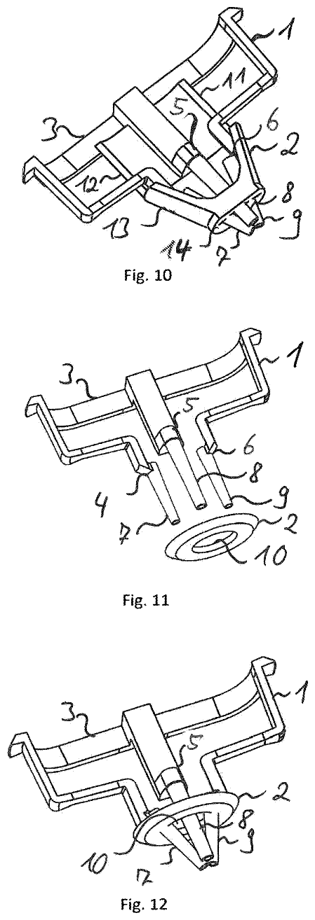

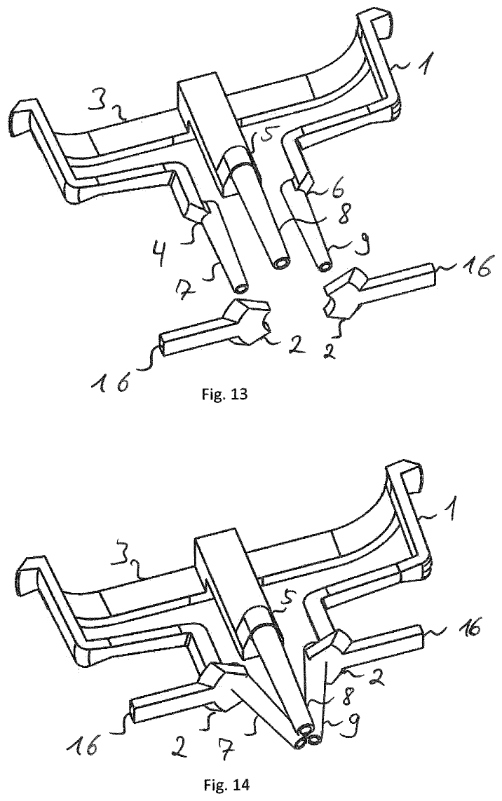

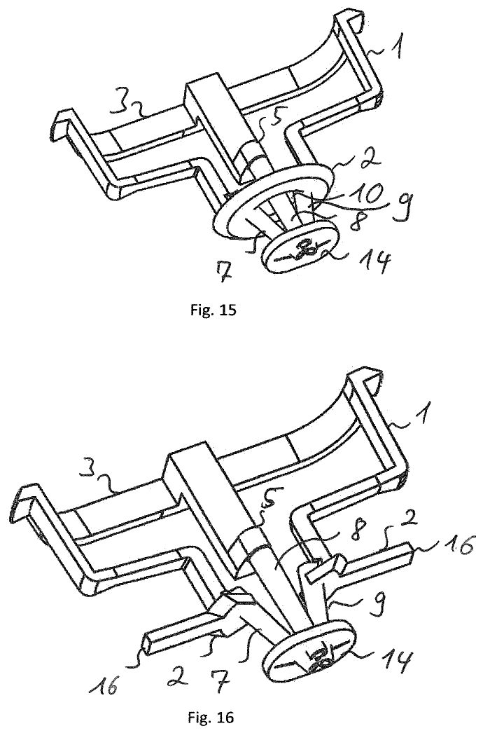

FIG. 12 a view corresponding to FIG. 12 with the lower part in place;

FIG. 13 a view corresponding to FIG. 7 with two-part clamping system;

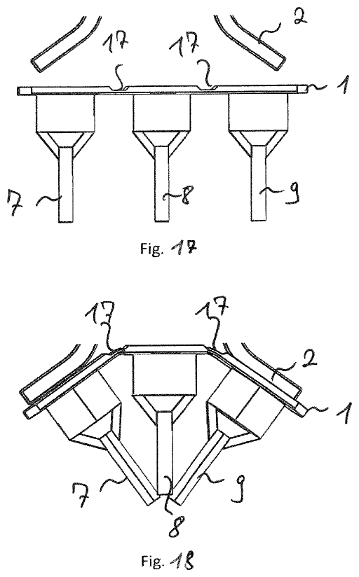

FIG. 14 a view corresponding to FIG. 13 with the two-part clamping system attached;

FIG. 15 a view corresponding to FIG. 12 with the disc-shaped lower part and cleaning element in place;

FIG. 16 a view corresponding to FIG. 14 with the two-part clamping system and cleaning element attached;

FIG. 17 a schematic sectional view of the first part and the second part of the sample receiving device in the separated state;

FIG. 18 a view corresponding to FIG. 17 in the assembled state

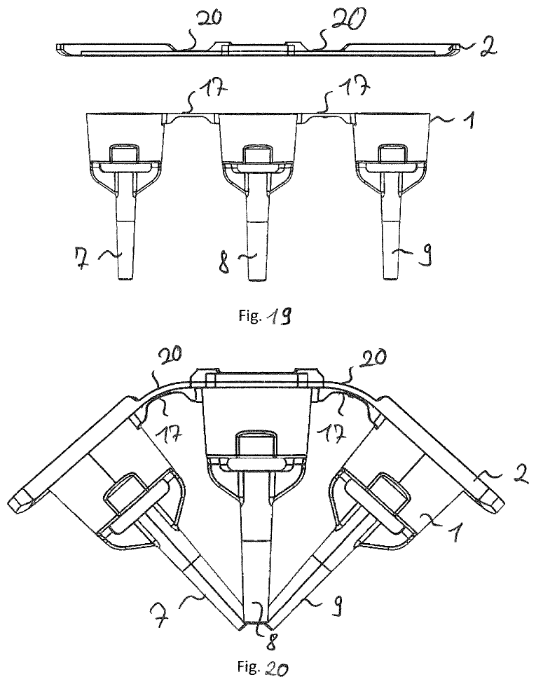

FIG. 19 a diagram corresponding to FIG. 17 of a further embodiment of the sample receiving device;

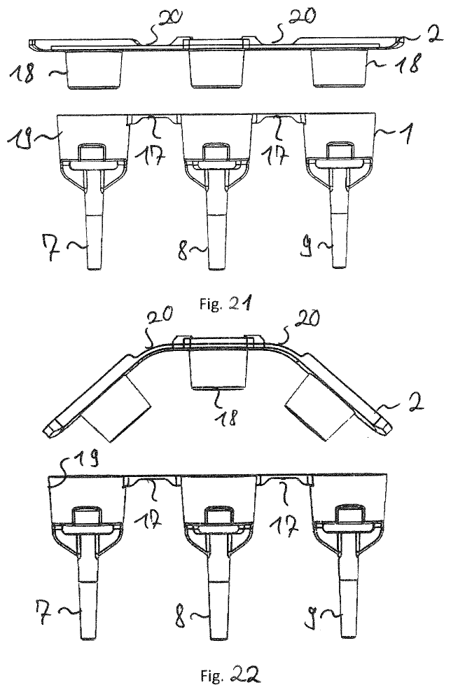

FIG. 20 a diagram corresponding to FIG. 18 of the embodiment shown in FIG. 3;

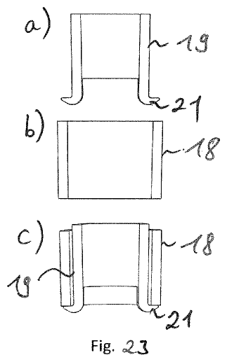

FIG. 21 a diagram corresponding to FIGS. 17 and 19 of a further embodiment of the sample receiving device;

FIG. 22 a diagram corresponding to FIG. 18 of the embodiment shown in FIG. 19, and

FIG. 23 an example of an embodiment with a connection mechanism.

FIG. 1 shows a sectional view of an upper part 1 of a sample receiving device. In the embodiment shown, the one-piece upper part 1 consists of a holding housing 3 made of a plastic material that is transparent to electromagnetic radiation in the optically visible wavelength range between 400 nm and 700 nm, i.e. at least 90 percent of the incident electromagnetic radiation is transmitted through the upper part 1. Alternatively, however, the upper part 1 can also be formed from an opaque material which has a transmittance of less than 30° percent of the incident electromagnetic radiation in the optically visible wavelength range. Three sample receiving units 7, 8, 9 are arranged on the holding housing 3, facing away from it, and are each connected to the holding housing 3 via a flexible connection 4, 5, 6, i.e. each via a connection point.

In the embodiment example shown, the sample receiving units 7, 8, 9 and the holding housing 3 are formed from the same material, but it may also be provided that different materials are used or that the material used differs from one another with respect to a property, for example its transparency, in the different elements.

The sample receiving units 7, 8, 9 are elongated or rod-shaped and are hollow inside, i.e. hollow cylinders or tubes. In further embodiments, however, the sample receiving units 7, 8, 9 or at least one of the sample receiving units 7, 8, 9 may also be in the form of a swab, brush, spatula, spatula knife, needle, membrane, rod, helical rod, fiber, hollow fiber, scoop, spatula, spoon, sponge, or foam. In addition, not all of the sample receiving units 7, 8, 9 need to be of the same type; it may also be provided that at least one of the sample receiving units 7, 8, 9 is of a different type from the other sample receiving units 7, 8, 9. On their side facing away from the holding housing 3 and facing a side to be picked up and subsequently analyzed, the sample receiving units in the illustrated embodiment example each have a smaller diameter than on their side facing the holding housing 3. The holding housing 3 itself is open on its side facing away from the sample receiving units 4, 5,6, but may also be closed in further embodiment examples.

Via the flexible, i.e. bendable and stretchable connections 4, 5, 6, the sample receiving units 7, 8, 9 can be bent or rotated to a small extent, the connections 4, 5, 6 are thus hinge-shaped. In addition, it is also possible to separate the sample receiving units 7, 8, 9 from the holding housing 3 by bending them beyond the respective bending radius, i.e. to separate the connections 4, 5, 6. It may also be provided that the connections 4, 5, 6 or at least one or two of the connections 4, 5, 6 are designed in such a way that they are moved back to the original position, i.e. the position reproduced in FIG. 1, when the lower part 2 is removed.

In the embodiment shown in FIG. 1, the two laterally arranged sample receiving units 7 and 9 are shorter than the centrally arranged sample receiving unit 8, i.e. their length is less than the length of the central sample receiving unit 8. In addition, the ends of the two laterally arranged sample receiving units 7 and 9 facing away from the holding housing 3 are arranged in a common plane, whereby the end of the central sample receiving unit 8 is not located in this plane, but in a plane closer to the holding housing 3. The sample receiving units 7, 8, 9 can each be of identical design, but it is also possible to use different sample receiving units 7, 8, 9 in pairs. The number is also variable and, in addition to the three sample receiving units 7, 8, 9 shown, can include only two sample receiving units or more than two sample receiving units, for example up to ten sample receiving units. The length of the sample receiving units is usually between 1 mm and 20 mm, preferably in the range of 8 mm and 14 mm.

The sample receiving units 7, 8, 9 shown are each designed as capillaries for holding liquid samples, for example drops of a specific liquid to be analyzed, such as drops of blood from a fingertip, in the range of milliliters, and can preferably hold sample volumes of 0.25 μl to 25 μl, particularly preferably 1 μl to 20 μl. Irrespective of the exact number of sample receiving units 7, 8, 9, each of the sample receiving units 7, 8, 9 generally has an identical internal volume or an internal cavity with an identical volume, i.e. an equally large sample volume can be held in each of the sample receiving units 7, 8, 9. In further embodiments, however, the sample volume of at least one of the sample receiving units 7, 8, 9 may also differ from the volume of the other sample receiving units 7, 8, 9. However, samples present as solids, for example as granules, powder or other bulk material, or samples in gel form can also be accommodated. Although the sample receiving units 7, 8, 9 are rigid in the embodiment shown, they may themselves be bendable in further embodiments.

FIG. 2 shows a top view of the described upper part 1, whereby the upper part 1 can now be seen from below, i.e. quasi from the perspective of the sample to be picked up. Recurring elements are provided with identical reference numerals in this Figure and also in the following Figures. The sample receiving units 7, 8, 9 are arranged in a row arrangement, i.e. their lower tips form an isosceles triangle with an angle at the sample receiving unit 8 greater than 120°.

FIG. 3 shows in a view corresponding to FIG. 1 the upper part 1 with joined sample receiving units 7, 8, 9, i.e. these are each bent at their web-shaped connections 4, 5, 6 and guided together with their tips in such a way that they are in direct contact with each other. In this position, in which all tips are also at the same height, i.e. all tips end in a single plane, a sample can be taken and a partial sample or aliquot can be contained in each of the sample receiving units 7, 8, 9.

FIG. 4 shows in a view corresponding to FIG. 2 the combined state shown in FIG. 3, in which practically a common suction tip is formed by the three tips of the sample receiving units 7, 8, 9.

A one-piece or monolithic lower part 2 matching the upper part 1 shown in FIGS. 1 to 4 is shown in a perspective view in FIG. 5. The housing 11 of the lower part 2 is also made of the material of the upper part 1 and has a straight first part 12 and a tapered second part 13 which is connected to the first part 12 in a form-fitting manner. The first part 12 is hollow in its interior and has a length which, in the embodiment shown, corresponds to the length of the sample receiving units 7, 8, 9, so that they can be received in the interior of the first part 12 without changing their position relative to each other. The first part 12 may be formed as tapering in the direction of the second part 13, but in this case a pitch angle of the taper is smaller than a corresponding pitch angle in the second part 13. In the embodiment example shown in FIG. 4, the pitch angle of the first part 12 is 0° (but could be up to 5°) due to its rectilinear design, while the pitch angle of the second part 13 is 45°.

The second part 13 also has a cavity inside and a final elongated opening 10, resulting in a through-opening of the housing 11, i.e. a lower part 2 open on two sides. However, the second part 13 is conically tapered in the direction of the opening 10. In the housing 11, a recess 15 is provided laterally in the first part 12, which can be plugged onto a corresponding guide part of the upper part 1, so that the lower part 2 can be displaced along this guide and can be countersunk in the upper part 1. When performing such a translational movement between the upper part 1 and the lower part 2, the tips of the sample receiving units 7, 8, 9 are moved towards each other by the second part 13 of the lower part 2 and finally emerge again at the opening 10, resulting in a defined position of the sample receiving units 7, 8, 9 for sample reception.

In FIG. 6, the lower part 2 is shown in a sectional view corresponding to FIG. 5 for clarification. Here, the diameter decreasing in the direction of the opening 10 can now be seen more clearly, through which the tips of the sample receiving units 7, 8, 9 are guided towards each other for sliding along the inner surface. For this purpose, the inner surface of the second part 13 is smooth, i.e. stepless, to allow resistance-free sliding of the tips.

FIG. 7 shows an assembled state of the lower part 2 and the upper part 1 in a perspective sectional view. The lower part 2 is placed on the upper part 1 in such a way that the first part 12 just encloses the sample receiving units 7, 8, 9, but does not move them from their initial position in which the sample receiving units 7, 8, 9 are spaced apart.

The condition after moving the lower part 2 in the direction of the upper part 1 or vice versa is shown in FIG. 8 in a view corresponding to FIG. 7. In this case, the lower part 2 is inserted into a recess in the upper part 1 so that the first part 12 lies inside the holding housing 3 and is enclosed by it, while the second part 13 lies outside the holding housing 3. In this pushed-together state, the tips of the sample receiving units 7, 8, 9 protrude from the opening 10 and form a common suction tip, which is also mechanically fixed by the opening 10. The lower part 2 and the upper part 1 can thus be connected to each other in a form-fit or force-fit connection. In addition, a stop can be provided on the upper part 1 or the lower part 2 to stop the translational movement between both parts 12 and 13 at a defined position.

Thus, a sample receiving device is provided as a consumable for typically single use, which can passively collect partial samples or aliquots from a single sample in a single step, manually or automatically. The partial samples taken can then be further analyzed. The fact that the various sample receiving units 7, 8, 9 can also be separated from the holding housing 3 means that each of the partial samples can be further treated and analyzed separately, thereby improving a diagnostic potential. The device described allows even small sample quantities to be taken with as little change as possible to the sample substance, while achieving a high degree of homogenization of the partial samples by taking them from a single original sample. In addition, unnecessary stress on the patient due to multiple sample collection is avoided, for example when taking samples from a fingertip. Due to an appropriate design of the sample receiving units 7, 8, 9, for example as capillaries, the device does not require active components such as complex microfluidics and can, if necessary, be easily operated by hand without the need for further complicated devices.

FIG. 9 shows, in a view corresponding to FIG. 5, a further embodiment of the lower part 2, in which the opening 10 is now covered by a cleaning element 14, such as a sponge, which fills at least the opening 10 and is arranged inside the housing 11 or outside the housing 11. The cleaning element 14 can alternatively or additionally be arranged in the second part 13 or in the first part 12. The cleaning element 14 can also be a single piece, i.e. consisting of one piece or monolithic, but it can also be formed from several individual parts, which can, however, also be connected to one another, for example welded together. The cleaning element 14 moves along and cleans an outer side of the sample receiving units 7, 8, 9 when performing the described relative movement of the upper part 1 and lower part 2. Accordingly, cleaning is also performed after sample reception when the lower part 2 and the upper part 1 are pulled apart, and the sample receiving units 7, 8, 9 are spatially spaced from each other with the sample picked up therein and the outer sides cleaned. FIG. 10 accordingly shows the collapsed state in a view corresponding to FIG. 8.

The volume-variable cleaning element 14 is particularly advantageous to use when a user of the sample receiving device may have special training for working in a laboratory and, accordingly, is insufficiently or not at all familiar with rules for clean sample reception. In the case of samples that are time-critical with regard to analysis due to ongoing biological or chemical processes, for example urine, a faster analysis can be performed because cleaning of the outside is already carried out during stripping. Furthermore, the cleaning element 14 also avoids contamination of the inner wall of the lower part 2 and, of course, of the outer sides of the sample receiving units 7, 8, 9, so that the reproducibility is increased due to clean sample containers and with a higher probability only the desired sample is contained in the sample receiving units 7, 8, 9 and also the safety for a user is increased, since an involuntary contact with the sample is avoided.

FIG. 11 shows a perspective view corresponding to FIG. 7 of the upper part 1 already described, onto which, however, a disk-shaped or ring-shaped lower part 2 is now to be fitted. For this purpose, the disc-shaped lower part 2 has an opening 10 which is arranged centrally as before, but a height of the lower part 2 is now significantly less than its length or width, typically the height is at most 10 percent of the length or width. The sample receiving units 7, 8, 9 are moved along the connections 4, 5, 6 by an external force, for example manually or by another machine, to the desired position in which their tips touch each other, and are locked or fixed in this position by being inserted into the opening 10, i.e. by placing the lower part 2 on top. Here, in turn, a form-fitting or force-fitting connection can be made between the upper part 1 and the lower part 2. FIG. 12 shows the upper part 1 with the attached lower part 2 from the embodiment described in FIG. 11.

As a further form of locking device, a holding device in the form of two clamps 16 can also be used as the lower part 2, as shown in FIG. 13 in a perspective view corresponding to FIG. 7. In this case, the sample receiving units 7, 8, 9 are moved to the desired position by an external force, as in the embodiment example reproduced in FIGS. 11 and 12, and are fixed in this position by the two clamps 16, as shown in a corresponding view in FIG. 14. A particular advantage of this design is that different distances between the sample receiving units 7, 8, 9 can be set. In further embodiments, only a single clamp 16 may be used, or the two clamps may be of one-piece design, i.e. comprising only a single part.

FIGS. 15 and 16 reproduce the embodiments shown in FIGS. 12 and 14, but now the cleaning element 14 is placed on the tips of the sample receiving units 7, 8, 9. For this purpose, the cleaning element 14 does not have to be integrated in the lower part 2, but can also be present as a separate component.

FIG. 17 shows a schematic sectional view of a first part 1 of a sample receiving device (for example a lower part) together with a second part 2 (for example an upper part). The first part 1 has the three rod-shaped sample receiving units 7, 8 and 9 arranged side by side. A bendable connection 17 in the form of a hinge is arranged centrally between each of two adjacent sample receiving units 7, 8, and 9. In the basic state shown in FIG. 17, the ends or tips of the sample receiving units 7, 8, and 9 facing away from the bendable connections 17, i.e., the ends facing in the direction of a sample to be received, are spatially spaced apart.

In the embodiment shown in FIG. 17, the sample receiving units 7, 8 and 9 are arranged in a row arrangement and the side of the sample receiving units 7, 8 and 9 facing the sample are arranged in a plane. The sample receiving units 7, 8 and 9 have an identical length, but in further embodiments can also be designed in such a way that at least one of the sample receiving units 7, 8 and 9 has a different length from the other sample receiving units 7, 8, 9. Similarly, the side of the first part 1 facing away from the sample is arranged in a plane in the basic state. The side of the sample receiving units 7, 8, 9 facing away from the sample (i.e., the side on which the bendable connection 17 is also arranged) is closed, while the sample receiving units 7, 8, 9 are designed as capillaries and have an opening on their side facing the sample. The number of sample receiving units 7, 8, 9 is also variable and, in addition to the three sample receiving units 7, 8, 9 shown, may include only two sample receiving units or more than two sample receiving units, for example up to ten sample receiving units. The length of the sample receiving units 7, 8, 9 is usually between 1 mm and 20 mm, preferably in the range of 8 mm and 14 mm.

The sample receiving units 7, 8, 9 shown are each designed as capillaries for holding liquid samples, for example drops of a specific liquid to be analyzed, such as drops of blood from a fingertip, in the range of milliliters, and can preferably hold sample volumes of 0.25 μl to 25 μl, particularly preferably 1 μl to 20 μl. Irrespective of the exact number of sample receiving units 7, 8, 9, each of the sample receiving units 7, 8, 9 generally has an identical internal volume or an internal cavity with an identical volume, i.e. an equally large sample volume can be held in each of the sample receiving units 7, 8, 9. In further embodiments, however, the sample volume of at least one of the sample receiving units 7, 8, 9 may also differ from the volume of the other sample receiving units 7, 8, 9. However, samples present as solids, for example as granules, powder or other bulk material, or gel-like samples can also be received. Although the sample receiving units 7, 8, 9 are rigid in the embodiment shown, they may themselves be bendable in further embodiments.

In the embodiment shown in FIG. 17, the second part 2 is made of the same material as the first part 1, but in further embodiments it can also be made of a different material. The second part 2 has a locking device which is designed in such a way that when the first part 1 is placed with its side facing away from the sample receiving units 7, 8, 9 on the second part 2, the bendable connection 17 is bent by the shape of the locking device, in the example shown a funnel-shaped recess, and the sample receiving units 7, 8, 9 are angled towards each other.

As shown in FIG. 18 in a view corresponding to FIG. 17, in the assembled state the first part 1 and the second part 2 are in direct contact with each other and the tips or ends of the sample receiving units 7, 8, 9 pointing in the direction of the sample to be received (or at least partial volumes thereof or an aliquot) are in direct contact with each other. At the intersection of the longitudinal axes of the rod-shaped sample receiving units 7, 8, 9, the sample to be picked up, typically a liquid, may be located and from there enter the capillaries. The form-fitting connection of the first part 1 and the second part 2 fixes the sample receiving units 7, 8, 9 in position.

As already explained, the sample receiving units 7, 8, 9 are elongated or rod-shaped and are hollow inside, i.e. capillaries or hollow cylinders or tubes. In further embodiments, however, the sample receiving units 7, 8, 9 or at least one of the sample receiving units 7, 8, 9 may also be in the form of a swab, brush, spatula, spatula knife, needle, membrane, rod, helical rod, fiber, hollow fiber, scoop, spatula, spoon, sponge, or foam. In addition, not all of the sample receiving units 7, 8, 9 need to be of the same type; it may also be provided that at least one of the sample receiving units 7, 8, 9 is of a different type from the other sample receiving units 7, 8, 9. On their side facing away from the bendable connection 17 and facing a sample to be picked up and subsequently analyzed, the sample receiving units 7, 8, 9 each have a smaller diameter in the illustrated embodiment than on their side facing the bendable connection 17.

Via the flexible, i.e. bendable and stretchable connections 17, the sample receiving units 7, 8, 9 can be bent or rotated to a small extent, the connections 17 are thus hinge-shaped. In addition, it is also possible to separate the sample receiving units 7, 8, 9 from each other by bending them beyond the respective bending radius, i.e. to break the connections 17. It may also be provided that the connections 17 or at least one or two of the connections 17 are configured in such a way that they are moved back to the original position, i.e. the position reproduced in FIG. 1, when the first part 1 is removed from the second part 2.

In FIG. 19, a side view corresponding to FIG. 17 shows another embodiment in which the first part 1 has three rod-shaped sample receiving units 7, 8 and 9 arranged side by side. A bendable connection 17 in the form of a hinge is again arranged centrally between two adjacent ones of the sample receiving units 7, 8 and 9. In this case, the second part 2 is designed as a bendable plate on which bendable areas 20 are arranged corresponding to the bendable connections 17, which can have the same mechanical properties as the bendable connections 17 of the first part 1. In FIG. 20, in a view corresponding to FIG. 18, the sample receiving device is shown in the assembled state, i.e., the first part 1 and the second part 2 are connected to each other, with the bendable connections 17 and the bendable areas 20 each being bent externally and by friction, an adhesive connection, or a combination of both, the first part and the second part are held together.

FIG. 21 shows, in a view corresponding to FIGS. 1 and 3, a further embodiment in which the second part, in contrast to the embodiment shown in FIG. 3, has bolt-shaped projections 18 which can be inserted into corresponding recesses of the first part 1. FIG. 22 shows the preformed second part 2, onto which the first part 1 can subsequently be placed and again fixed with an adhesive connection and/or a connection held by friction between the projections 18 and the recesses into which they are inserted.

FIG. 23 shows a possible design of this recess 19 in a lateral sectional view in FIG. 23b), while in FIG. 23a) the bolt-shaped projection 18 is shown as a hollow body. In FIG. 23c), both elements are assembled, with a stop 21 on recess 19 now limiting the movement of recess 18. Thus, in the illustrated embodiment example, the second part 2 is inserted into the first part 1.

Thus, a sample receiving device is provided as a consumable for typically single use, which can passively collect partial samples or aliquots from a single sample in a single step, manually or automatically. The partial samples taken can then be further analyzed. Due to the fact that the different sample receiving units 7, 8, 9 can also be separated from each other, each of the partial samples can be further treated and analyzed separately, thus improving a diagnostic potential. The device described allows even small sample quantities to be taken with as little change as possible to the sample substance, while achieving a high degree of homogenization of the partial samples by taking them from a single original sample. In addition, unnecessary stress on the patient due to multiple sample collection is avoided, for example when taking samples from a fingertip. Due to an appropriate design of the sample receiving units 7, 8, 9, for example as capillaries, the device does not require active components such as complex microfluidics and can, if necessary, be easily operated by hand without the need for further complicated devices.

The described device or a corresponding sample collection method with said sample receiving device can be used in the medical or veterinary field, but also industrially or for the analysis of food such as beverages or ecological analysis. The device described is particularly advantageous in that both sample loss (which should generally be avoided), sample smearing (which can lead to erroneous measurement results), and user exposure to potentially infectious or otherwise harmful samples are completely or largely avoided.

In particular, the invention also relates to the following aspects:

In a first aspect, a sample receiving device having an upper part 1 and a lower part 2, which can be connected to one another in a form-fitting and/or force-fitting manner, the upper part 1 having a holding housing 3, at least two rod-shaped sample receiving units 7, 8, 9 or at least two bendable sample receiving units 7, 8, 9 being arranged on the holding housing 3 and connected thereto in each case via a bendable connection 4, 5, 6, which sample receiving units 7, 8, 9 are arranged spatially spaced apart from one another in a basic state, the lower part 2 having a locking device which is designed in such a way that the at least two sample receiving units 7, 8, 9 can be guided towards one another either by an external force or by the locking device of the lower part 2, so that the sides of the at least two sample receiving units 7, 8, 9, which are arranged pointing in the direction of a sample, are arranged directly next to one another, are locked in this position by the locking device and a single sample can be received in the at least two sample receiving units 7, 8, 9 or with the at least two sample receiving units 7, 8, 9.

In a second aspect, a sample receiving device according to the first aspect, wherein the lower part 2 comprises a housing 11 provided with an opening 10 serving as a locking device.

In a third aspect, a sample receiving device according to the second aspect, wherein the sample receiving units 7, 8, 9 are insertable into the opening 10 of the housing 11 and are movable relative to each other by the external force such that the sample receiving units 7, 8, 9 are locked in the opening 10 serving as a locking device after insertion.

In a fourth aspect, a sample receiving device according to any one of aspects 1-3, wherein the lower part (2) comprises a housing 11 provided with a through opening 10 serving as a locking device, the housing 11 having at least one tapered part 13, wherein the lower part 2 can be placed on the at least two sample receiving units 7, 8, 9 in a translational movement in such a way that the at least two sample receiving units 7, 8, 9, when introduced into the tapered part 13 of the lower part 2, are guided towards each other by the tapered shape on their sides which are arranged pointing in the direction of a sample.

In a fifth aspect, a sample receiving device according to the fourth aspect, wherein the tapered portion of the lower part 2 is configured as a conical tapered portion 13.

In a sixth aspect, a sample receiving device according to any one of aspects 2-5, wherein the lower part 2 comprises a rectilinearly extending part 12 and the rectilinearly extending part 12 of the lower part 2 is insertable into a corresponding recess of the upper part 1 for establishing operational readiness of the at least two sample receiving units 7, 8, 9 during the translational movement.

In a seventh aspect, a sample receiving device according to any one of aspects 2-6, wherein a length of the rectilinearly extending part 12 of the lower part 2 corresponds to a length of the sample receiving units 7, 8, 9 and/or the length of the sample receiving units 7, 8, 9 is selected such that the side of the sample receiving units 7, 8, 9 facing in the direction of the sample is arranged in the region of the opening 10 provided on the lower part 2.

In an eighth aspect, a sample receiving device according to any one of aspects 2-7, wherein the lower part 2 is movably guided in a guide arranged in the holding housing 3 of the upper part 1 and is preferably limited in its ability to move by at least one stop.

In a ninth aspect, a sample receiving device according to the first aspect, wherein the lower part 2 comprises a preferably two-part holding device as a locking device by which the at least two sample receiving units 7, 8, 9 moved by the external force are clamped in position.

In a tenth aspect, a sample receiving device according to any one of the preceding aspects, wherein at least one of the sample receiving units 7, 8, 9 is in the form of a capillary, a tube, a fiber, preferably a hollow fiber, a swab, a brush, a spatula, a spatula knife, a needle, a membrane, a scoop, a spoon, a sponge, preferably a solid sponge, or a rod, preferably a helical rod, or is formed with a capillary, tube, fiber, preferably hollow fiber, swab, brush, spatula, spatula knife, needle, membrane, scoop, spoon, sponge, preferably solid sponge, or rod, preferably helical rod.

In an eleventh aspect, a sample receiving device according to one of the preceding aspects, wherein the upper part 1 comprises exactly three sample receiving units 7, 8, 9, which are arranged in a row arrangement and of which the sides of the two sample receiving units 7, 9 arranged at the edge facing away from the holding housing 3 are arranged in a first plane in the basic state and the side of the centrally arranged sample receiving unit 8 facing away from the holding housing 3 is arranged in a second plane, which is different from the first plane and is closer to the holding housing 3, in the basic state with respect to the two laterally arranged sample receiving units 7, 9.

In a twelfth aspect, a sample receiving device according to any one of the preceding aspects, wherein the lower part 2 comprises a volume-variable cleaning element 14 covering the opening 10, preferably a sponge, into which the at least two sample receiving units 7, 8, 9 are insertable or a volume-variable cleaning element 14 covering the opening 10 is placeable onto the at least two sample receiving units 7, 8, 9.

In a thirteenth aspect, a sample receiving device according to any of the preceding aspects, wherein the holding housing 3 is formed closed on a side facing away from the at least two sample receiving units 7, 8, 9.

In a fourteenth aspect, a method for sample collection by means of the sample receiving device according to any one of aspects 1-13, wherein the at least two sample receiving units 7, 8, 9 are guided towards each other either by an external force or by the locking device of the lower part 2, so that the sides of the at least two sample receiving units 7, 8, 9 arranged facing in the direction of a sample are arranged directly next to each other, are locked in this position by the locking device and a single sample is received in the at least two sample receiving units 7, 8, 9 or with the at least two sample receiving units 7, 8, 9.

In a fifteenth aspect, a method for sample collection, in which the lower part 2 of the sample receiving device according to one of aspects 4 to 8 or 10 to 13 is moved with respect to the upper part 1 in such a way that the at least two sample receiving units 7, 8, 9 are inserted into the tapered part 13 of the lower part 2 and sides of the at least two sample receiving units 7, 8, 9 arranged facing in the direction of a sample are guided towards each other so that the sides of the at least two sample receiving units 7, 8, 9 arranged facing in the direction of a sample are arranged directly next to each other and a single sample is collected in the at least two sample receiving units 7, 8, 9, 8, 9 are guided towards each other, so that the sides of the at least two sample receiving units 7, 8, 9 arranged facing in the direction of a sample are arranged directly next to each other and a single sample is received in the at least two sample receiving units 7, 8, 9.

Features of the various embodiments disclosed in the embodiment examples only may be combined and claimed individually.

1.-24. (canceled)

25. A sample receiving device, comprising:
a first part and a second part,
wherein the first part has at least two rod-shaped sample receiving units connected to one another via a bendable connection,
whose ends facing away from the bendable connection are spatially spaced apart from one another in a basic state,
wherein the second part has a locking device which is designed in such a way that, when the first part is placed with its side facing away from the sample receiving units onto the second part, the bendable connection is bent by the shape of the locking device and the ends of the at least two sample receiving devices can be guided towards one another so that
the sides of the at least two sample receiving units facing in the direction of a sample are arranged directly next to one another, are locked in this position by the locking device and a single sample can be received in the at least two sample receiving units or with the at least two sample receiving units.

26. The sample receiving device according to claim 25, wherein the bendable connection is a hinge-shaped connection.

27. The sample receiving device according to claim 25, wherein the bendable connection is arranged centrally between the two sample receiving units.

28. The sample receiving device according to claim 25, wherein at least one of the sample receiving units is in the form of a capillary, a tube, a fiber, preferably a hollow fiber, a swab, a brush, a spatula, a spatula knife, a needle, a membrane, a scoop, a spoon, a sponge, preferably a solid sponge, or a rod, preferably a helical rod, or has a capillary, a tube, a fiber, preferably a hollow fiber, a swab, a brush, a spatula, a spatula knife, a needle, a membrane, a scoop, a spoon, a sponge, preferably a solid sponge, or a rod, preferably a helical rod.

29. The sample receiving device according to claim 25, wherein the first part has precisely three sample receiving units which are arranged in a row arrangement and of which the sides of the sample receiving units facing the sample are arranged in a plane in the basic state, the one of the sample receiving units arranged centrally between two sample receiving units being connected to the respectively adjacent sample receiving unit via in each case one bendable connection.

30. The sample receiving device according to claim 25, wherein the first part is formed closed at a side facing away from the tips of the at least two sample receiving units arranged facing in the direction of the sample.

31. The sample receiving device according claim 25, wherein the locking device of the second part is funnel-shaped for receiving the first part.

32. The sample receiving device according to claim 25, wherein the first part and the second part can be connected to one another in a form-fitting or force-fitting manner.

33. The sample receiving device according to claim 25, wherein the first part with the at least two sample receiving units is formed from a plastic material which is transparent to electromagnetic radiation in the optically visible wavelength range.

34. The sample receiving device according to claim 25, further comprising an upper part as the first part and a lower part as the second part, which can be connected to one another in a form-fitting and/or force-fitting manner, wherein the upper part has a holding housing, the at least two rod-shaped sample receiving units or the at least two bendable sample receiving units being arranged on the holding housing and connected thereto in each case via a bendable connection,
which are spatially spaced from each other in a basic state,
wherein the lower part comprises a locking device which is designed in such a way that the at least two sample receiving units can be guided towards each other either by an external force or by the locking device of the lower part, so that the sides of the at least two sample receiving units facing in the direction of a sample are arranged directly next to one another, are locked in this position by the locking device and a single sample can be received in the at least two sample receiving units or with the at least two sample receiving units.

35. The sample receiving device according to claim 34, wherein the lower part comprises a housing provided with an opening serving as a locking device.

36. The sample receiving device according to claim 35, wherein the sample receiving units are insertable into the opening of the housing and are movable relative to each other by the external force in such a way that the sample receiving units are locked in the opening serving as a locking device after insertion.

37. The sample receiving device according to claim 34, wherein the lower part has a housing which is provided with a through-opening serving as a locking device and which has at least one tapered part, wherein the lower part can be placed on the at least two sample receiving units in a translational movement in such a way that the at least two sample receiving units, when introduced into the tapered part of the lower part, are guided towards each other by the tapered shape on their sides arranged pointing in the direction of a sample.

38. The sample receiving device according to claim 37, wherein the tapered part of the lower part is designed as a conical tapered part.

39. The sample receiving device according to claim 35, wherein the lower part comprises a rectilinearly extending part and the rectilinearly extending part of the lower part is insertable into a corresponding recess of the upper part for establishing operational readiness of the at least two sample receiving units during the translational movement.

40. The sample receiving device according to claim 35, wherein a length of the rectilinearly extending part of the lower part corresponds to a length of the sample receiving units and/or the length of the sample receiving units is selected such that the side of the sample receiving units facing in the direction of the sample is arranged in the region of the opening provided on the lower part.

41. The sample receiving device according to claim 35, wherein the lower part is movably guided in a guide arranged in the holding housing of the upper part and is limited in its ability to move by at least one stop.

42. The sample receiving device according to claim 35, wherein the lower part has a two-part holding device as a locking device, by which the at least two sample receiving units moved by the external force are clamped in their position.

43. The sample receiving device according to claim 25, wherein the upper part has exactly three sample receiving units which are arranged in a row arrangement and of which the sides facing away from the holding housing of the two sample receiving units arranged at the edges are arranged in a first plane in the basic state, and the side of the centrally arranged sample receiving unit facing away from the holding housing is arranged in a second plane, which is different from the first plane and closer to the holding housing, in the basic state with respect to the two laterally arranged sample receiving units.

44. The sample receiving device according to claim 25, wherein the lower part has a volume-variable cleaning element covering the opening, preferably a sponge, into which the at least two sample receiving units can be inserted or a volume-variable cleaning element covering the opening can be placed on the at least two sample receiving units.

45. The sample receiving device according to claim 25, wherein the holding housing is formed closed at a side facing away from the sides of the at least two sample receiving units arranged facing in the direction of the sample.

46. A method for receiving samples by the sample receiving device according to claim 25, in which the bendable connection, when the first part is placed with its side facing away from the ends of the sample receiving units onto the second part, is bent by the shape of the locking device and the ends of the at least two sample receiving units are guided towards each other, so that the ends of the at least two sample receiving units arranged pointing in the direction of a sample are arranged directly next to each other, are locked in this position by the locking device and a single sample can be received in the at least two sample receiving units or with the at least two sample receiving units.

47. A method for receiving samples by the sample receiving device of claim 34, in which the at least two sample receiving units are guided towards each other either by an external force or by the locking device of the lower part, so that the sides of the at least two sample receiving units are arranged directly next to one another, are locked in this position by the locking device, and a single sample is received in the at least two sample receiving units or with the at least two sample receiving units.

48. A method for receiving samples, in which the lower part of the sample receiving device according to claim 34 is moved with respect to the upper part in such a way that the at least two sample receiving units are introduced into the tapered part of the lower part and sides of the at least two sample receiving units arranged facing in the direction of a sample are guided towards one another so that the sides of the at least two sample receiving units facing in the direction of a sample are arranged directly adjacent to each other and a single sample is received in the at least two sample receiving units.
